(19) United States
(12) Patent Application Publication
(10) Pub. No.: US 2025/0051834 A1
(43) Pub. Date: Feb. 13, 2025

(54) Single-Cell Epigenomic Profiling Using Droplet Fluidics and Hydrogels

(71) Applicants: CZ BIOHUB SF, LLC, San Francisco, CA (US); THE REGENTS OF THE UNIVERSITY OF CALIFORNIA, Oakland, CA (US)

(72) Inventors: Adam Abate, Oakland, CA (US); Xiangpeng Li, Oakland, CA (US)

(21) Appl. No.: 18/718,585

(22) PCT Filed: Dec. 13, 2022
(86) PCT No.: PCT/US22/52670

§ 371 Date: Jun. 11, 2024

Related U.S. Application Data
(60) Provisional application No. 63/288,805, filed on Dec. 13, 2021.

(51) Int. Cl. C12Q 1/6841; C12N 5/00; C12Q 1/6806; G01N 33/53

(52) U.S. Cl. CPC C12Q 1/6841; C12N 5/0012; C12Q 1/6806; G01N 33/5308

(57) ABSTRACT

The present disclosure provides materials and methods for partitioning cells and high throughput, single-cell epigenetic sequencing. Methods for using chemically-modified hydrogels are also provided herein. Wherein determining the epigenomic state of a single-cell comprises preparing a functionalized hydrogel, wherein said functionalized hydrogel is chemically modified with a functional group capable of binding to a capture reagent.

### CROSS-REFERENCE TO RELATED APPLICATIONS

The present application claims priority to U.S. Provisional Patent Application No. 63/288,805, filed on Dec. 13, 2021, their entirety of which are incorporated by reference herein.

### FIELD

The present disclosure relates generally to methods for determining the epigenetic state of single cells.

### BACKGROUND

Single-cell sequencing technologies refer to the methods to obtain genomics, transcriptomics or multi-omics information of single cells. Traditional sequencing methods only work with samples of many cells, and are thus unable to resolve cellular heterogeneity.

Although several single-cell sequencing methods are available, there are many limitations. For example, microfluidics-based single-cell sequencing methods are technologically challenging for biologists to perform. Well plate-based methods lack sufficient throughput. As most available methods are targeted for transcriptome sequencing, single-cell genome sequencing and other multiomic technologies are not well established. Significantly, high-throughput single-cell epigenomic sequencing in a massively parallelized fashion that matches what is currently available for genomics, transcriptomics, and proteomics is unavailable. There thus exists a need in the art for high throughput, single-cell epigenomic sequencing.

### SUMMARY OF THE INVENTION

The present disclosure provides, in one embodiment, a method of determining the epigenomic state of a single cell comprising the steps of: (a) preparing a functionalized hydrogel, wherein said functionalized hydrogel is chemically modified with a functional group capable of binding to a capture reagent; (b) preparing a functionalized capture reagent, wherein said functionalized capture reagent is chemically modified with a functional group capable of binding to the functional group of the functionalized hydrogel of (a), and wherein said capture reagent is also capable of binding to a nucleic acid; (c) encapsulating single cells into particles comprising a cell lysis buffer and the functionalized hydrogel of (a) under conditions that allow cell lysis; (d) preparing a nucleic acid from the encapsulated single cells of (c) under conditions that allow binding of the capture reagent to (i) the nucleic acid, and (ii) the functional group of the functionalized hydrogel, thereby forming a captured nucleic; (e) preparing the captured nucleic acid of (d) for sequencing, wherein said preparing comprises universal adaptor ligation and barcoding; and (f) sequencing the captured nucleic acid and thereby determining the epigenomic state of a single cell.

In one embodiment, the hydrogel comprises a polysaccharide selected from the group consisting of agarose, alginate, chitin, chitosan, or hybridize hydrogel material containing a polysaccharide. In some embodiments, an aforementioned method is provided wherein the functional group capable of binding to the capture reagent is selected from the group consisting of azide (N₃), dibenzocyclooctyne (DBCO), alkyne, tetrazine (TZ), methyltetrazine, trans-cyclooctene (TCO), cyclooctene, norbornene (NZ), cyclopropene, thiol, bromo, tosylate, maleimide, amine, carboxylic acid and NHS ester.

In some embodiments, an aforementioned method is provided wherein the functional group capable of binding to the functional group of the functionalized hydrogel is selected from the group consisting of azide (N₃), dibenzocyclooctyne (DBCO), alkyne, tetrazine (TZ), methyltetrazine, trans-cyclooctene (TCO), cyclooctene, norbornene (NZ), cyclopropene, thiol, bromo, tosylate, maleimide, amine, carboxylic acid and NHS ester.

In other embodiments, an aforementioned method is provided wherein the capture reagent is selected from the group consisting of an antibody, streptavidin, avidin, and aptamers. In still other embodiments, an aforementioned method is provided wherein the nucleic acid is genomic DNA. In one embodiment, the capture reagent is capable of binding to an epigenetic marker of the genomic DNA. In another embodiment, the epigenetic marker is selected from the group consisting of a genomic DNA modification marker, a histone modification marker, a DNA-transcription factor interaction marker, a DNA accessibility marker, a chromatin conformation marker, and a mRNA-nucleosome interaction marker. In yet another embodiment, the epigenetic marker is a genomic DNA modification marker selected from the group consisting of 5-methylcytosin, 5-hydroxymethylcytosine, 5-formylcytosine, 5-carboxylcytosine, and 3-methylcytosine.

In other embodiments, an aforementioned method is provided wherein the conditions of (d) that allow binding of the capture reagent to (i) the nucleic acid, and (ii) the functional group of the functionalized hydrogel comprises cross-linking the capture reagent to the hydrogel.

### BRIEF DESCRIPTION OF THE DRAWINGS

FIG. 1 shows a workflow for sequencing DNA with a 5hmC epigenetic marker

FIG. 2 shows a workflow for sequencing DNA with a 5mC epigenetic marker

### DETAILED DESCRIPTION

The present disclosure provides materials and methods for addressing the aforementioned unmet need in the art by providing high-throughput workflows for single-cell epigenomic sequencing using a capture/profiling approach with hydrogel scaffolds, and thus facilitates a scale and form of epigenomic profiling beyond what has been previously reported. Current technologies are either very limited in the scope of what they are capable of, are only applicable to sequencing/profiling of bulk populations, and/or require laborious processing using 96-well plates that limits single-cell sample throughput. The present disclosure, on the other hand, provides a broadly applicable way to interrogate many different forms of epigenomic modifications at high-throughput, single-cell scales. The implementation of the present disclosure is compatible with other microfluidic techniques, as will be understood by those of skill in the art.

As described herein, the present disclosure provides methods that use hydrogel beads which capture, barcode, and process (e.g., Next Generation Sequencing) nucleic acids or proteins to determine the epigenomic state of single cells. Capturing and lysis of individual cells that are immobilized in a hydrogel matrix, thus entrapping genomic DNA, is contemplated. In some embodiments, the hydrogel is optionally modified with chemical handles that facilitate the capture of DNA through, for example, one of two approaches: 1) The use of biochemical modification of DNA (e.g., Bisulfide, APOBEC, TET2, or T4-beta-GT treatment) to tag epigenomic modifications; and/or 2) the use of affinity reagents that either directly or indirectly bind DNA of interest (e.g., antibodies binding CpG DNA, modified histones, transcription factors, or RNA polymerase) akin to the method of chromatin immunoprecipitation.

Grosselin et al. report a high-throughput droplet microfluidics platform to profile chromatin landscapes of thousands of cells at single-cell resolution (Grosselin, K., et al., Nat Genet 51, 1060-1066 (2019), and Rotem et al., report a combination microfluidics, DNA barcoding and sequencing to collect chromatin data at single-cell resolution (Rotem et al., Nat Biotechnol 33, 1165-1172 (2015)), however these methods could not directly measure the modification of bases of the genomic DNA or histone modifications, such as 5-mC or 5hmC.

The present disclosure provides methods and compositions for high throughput single-cell epigenetic sequencing that is simple to operate. The present disclosure provides a rapid method of high-throughput, single-cell epigenetic sequencing using single-cell partitioning techniques described herein.

The methods and compositions provided herein provide several key innovations over existing technologies. First, in one embodiment of the disclosure, single cells are isolated and encapsulated in hydrogel microbeads, optionally by shaken emulsification. The hydrogel microbeads are size-selected based on buoyancy and centrifugation force. This embodiment allows for fast processing of millions of cells without any complex instrumentation such as microfluidics or fluorescence-activated cell sorting (FACS) and at a throughput surpassing other available methods.

Second, in another embodiment, the single cells embedded with in hydrogel microbeads are lysed and washed in solution. Because the hydrogel materials allow free diffusion of any molecules with hydraulic diameters smaller than the pore size, but sterically trap genomic DNA, this invention allows multi-step molecular biology reactions required for genomic sequencing which are not easily performed in other systems. Also, the existing single-cell analysis platform such as microwell, microbeads, or microfluidic-based barcoding methods lack the ability to perform multi-step reactions or the workflows are long and challenging to perform.

Third, in still another embodiment, the present disclosure enables single-cell epigenetic sequencing. In some embodiments, the hydrogel is modified with chemical handles that facilitate the capture of DNA through, for example, one of two approaches: 1) The use of biochemical modification of DNA (e.g., Bisulfide, APOBEC, TET2, or T4-beta-GT treatment) to tag epigenomic modifications; and/or 2) the use of affinity reagents that either directly or indirectly bind DNA of interest (e.g., antibodies binding CpG DNA, modified histones, transcription factors, or RNA polymerase) akin to the method of chromatin immunoprecipitation.

In some embodiments, to the genomes trapped in hydrogel microsphere, chemically labelled antibodies or affinity probes targeting specific epigenetic markers may be introduced, which specifically bind to the corresponding epigenetic markers and chemically crosslink with the hydrogel network. After genomic DNA fragmentation and washing, only retaining DNA fragments are the ones captured by antibodies or affinity probes. The DNA fragments are then barcoded using microfluidics for sequencing library preparation. This high throughput single cell epigenetic sequencing method can be applied to several epigenetic markers, which include but not limited to, genomic DNA modification (5-methylcytosin, 5-hydroxymethylcytosine, 5-formylcytosine, 5-carboxylcytosine, and 3-Methylcytosine), histone modifications, DNA-transcription factor interaction, DNA accessibility, chromatin conformation, and mRNA-nucleosome interaction.

Among the numerous improvements and advantages provide herein, the present disclosure provides methods that require minimal instrumentation, significantly lowering the technical expertise required to deploy single-cell whole genome sequencing, single-cell epigenetic sequencing of bacteria and fungi on commercial platforms designed for mammalian cells, and (other) multi-omic single-cell sequencing including genomics, transcriptomics, and proteomics.

As used herein, the term “sample” or “biological sample” encompasses a variety of sample types obtained from a variety of sources, which sample types contain biological material. For example, the term includes biological samples obtained from a mammalian subject, e.g., a human subject, and biological samples obtained from a food, water, or other environmental source, etc. The definition encompasses blood and other liquid samples of biological origin, as well as solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides. The term “sample” or “biological sample” encompasses a clinical sample, and also includes cells in culture, cell supernatants, cell lysates, cells, serum, plasma, biological fluid, and tissue samples. “Sample” and “biological sample” includes cells, e.g., bacterial cells or eukaryotic cells; biological fluids such as blood, cerebrospinal fluid, semen, saliva, and the like; bile; bone marrow; skin (e.g., skin biopsy); and viruses or viral particles obtained from an individual.

As described more fully herein, in various aspects the subject methods may be used to detect and/or quantify a variety of components from such biological samples. Components of interest include, but are not necessarily limited to, cells (e.g., circulating cells and/or circulating tumor cells), viruses and viral genomes, polynucleotides (e.g., DNA and/or RNA), polypeptides (e.g., peptides and/or proteins), and many other components that may be present in a biological sample. As described herein, the present disclosure provides methods and compositions for detecting and quantitating materials from single cells. In one embodiment, the epigenetic state of the cells of a sample are determined as described herein.

The terms “polynucleotide” and “nucleic acid” and “target nucleic acid” refer to a polymer composed of a multiplicity of nucleotide units (ribonucleotide or deoxyribonucleotide or related structural variants) linked via phosphodiester bonds. A polynucleotide or nucleic acid can be of substantially any length, typically from about six (6) nucleotides to about 10⁹ nucleotides or larger. Polynucleotides and nucleic acids include RNA, cDNA, genomic DNA. In particular, the polynucleotides and nucleic acids of the present invention refer to polynucleotides encoding a chromatin protein, a nucleotide modifying enzyme and/or fusion polypeptides of a chromatin protein and a nucleotide modifying enzyme, including mRNAs, DNAs, cDNAs, genomic DNA, and polynucleotides encoding fragments, derivatives and analogs thereof. Useful fragments and derivatives include those based on all possible codon choices for the same amino acid, and codon choices based on conservative amino acid substitutions. Useful derivatives further include those having at least 50% or at least 70% polynucleotide sequence identity, and more preferably 80%, still more preferably 90% sequence identity, to a native chromatin binding protein or to a nucleotide modifying enzyme.

The term “oligonucleotide” refers to a polynucleotide of from about six (6) to about one hundred (100) nucleotides or more in length. Thus, oligonucleotides are a subset of polynucleotides. Oligonucleotides can be synthesized manually, or on an automated oligonucleotide synthesizer (for example, those manufactured by Applied BioSystems (Foster City, CA)) according to specifications provided by the manufacturer or they can be the result of restriction enzyme digestion and fractionation.

“Epigenetics” or epigenome” or “epigenetic markers” are terms that describe the heritable changes in gene expression patterns that are independent of primary DNA sequence changes and affect the outcome of a locus or chromosome without altering the underlying DNA sequence. As used herein, the epigenetic marker can be a genomic DNA modification marker, a histone modification marker, a DNA-transcription factor interaction marker, a DNA accessibility marker, a chromatin conformation marker, and a mRNA-nucleosome interaction marker. As described herein, exemplary epigenetic markers include, but are not limited to, 5-methylcytosin, 5-hydroxymethylcytosine, 5-formylcytosine, 5-carboxylcytosine, and 3-methylcytosin.

Generally, other nomenclature used herein and many of the laboratory procedures in cell culture, molecular genetics and nucleic acid chemistry and hybridization, which are described below, are those well-known and commonly employed in the art. (See generally Ausubel et al. (1996) supra; Sambrook et al, Molecular Cloning: A Laboratory Manual, Second Edition, Cold Spring Harbor Laboratory Press, New York (1989), which are incorporated by reference herein). Standard techniques are used for recombinant nucleic acid methods, polynucleotide synthesis, preparation of biological samples, preparation of cDNA fragments, isolation of mRNA and the like. Generally enzymatic reactions and purification steps are performed according to the manufacturers' specifications.

The present disclosure provides methods and materials for epigenetic sequencing one or more nucleic acids from a single cell. The methods provided herein comprising encapsulating cells in permeable compartments without microfluidic control. In some embodiments, the permeable compartments are achieved by (1) encapsulating the cells in hydrogel microbeads, or (2) fixation and permeabilization of the cells. Single cells are then barcoded using methods and compositions provided herein.

Hydrogel-based compartmentalization comprises, in some embodiments, mixing cells with gel precursor materials, adding an immiscible carrier, and agitating the mixture. The agitation can comprise passing the fluids through a constriction, such as a syringe needle or microchannel network, or by shaking the mixture in a reservoir, such as with a vortexer, homogenizer, or shaking the tube. The resultant emulsion will comprise a range of droplet sizes, some of which contain single cells. The loading rate of the cells can be controlled by adjusting cell concentration, dilution, and addition of precursor materials prior to agitation. Particle properties can be selected to facilitate this, for example, by controlling particle chemistry, porosity, and functionalization. Once encapsulated, the sample is solidified to produce particles. These particles can comprise hydrogels, polymers, plastics, glasses, etc. The resultant particles can be further processed to enable single cell sequencing, including particle size selection and cell analyte preparation. These steps can be done in any order optimal for the particular workflow. To facilitate processing, particles can be transferred between carrier phases using a number of techniques, such as chemical or electrical demulsification, solvent transfer, particle templated emulsification, etc.

To facilitate later steps involving barcoding, permeable compartments of optimal size can be selected from the polydisperse suspension. In some embodiments, this is achieved by filtering the suspension with a series of filters to select a desired size range. Alternatively, particles can be selected based on filtering or density gradient centrifugation, collecting or discarding appropriate layers. In general, particles of a size similar to mammalian cells are optimal for barcoding with instruments designed for mammalian cell sequencing. If other instruments are to be used that are designed for barcoding different samples, a different size particle can be selected, as optimal for the workflow. Other methods for selecting particles contemplated by the present disclosure involve the use of hydrodynamic forces, some of which involve microfluidics. For example pinched flow fractionation and inertial ordering are passive techniques for selecting desired particles. Flow cytometry, an active sorting technique, may also be used to select particles based on optical properties. This provides additional benefits, such as allowing cell contents to be analyzed and used to inform selection.

To facilitate access to cell-based analytes, such as nucleic acids, the cells encapsulated in the particles can be processed, for example to lyse cell walls or membranes, capture mRNA or proteins, and the like. In some embodiments, this step can be achieved with the particles in an immiscible (e.g. oil) or miscible (e.g. aqueous) carrier to facilitate transfer of necessary materials into and out of the particles. Reagents can be mixed with the particles to prepare cells and their biomolecules for analysis. For example, detergents, enzymes (e.g. lysozyme, proteinase K), can digest cell molecules to afford access to nucleic acids and digest molecules that could interfere with later steps, such as nucleases. In the case of eukaryotic cells, chromatin may be digested to facilitate access to genomic DNA. Other digestions can also be performed to facilitate analysis. For example, nuclease digestion can be used to fragment genomic DNA into pieces suitable for sequencing. Tagmentation can be used to fragment and add universal adaptors for barcoding and/or sequencing. Alternatively, cellular analytes can be amplified to facilitate their analysis. For example, genomic DNA from single cells can be subjected to whole genome amplification to provide multiple copies for later analysis which, according to some embodiments of the present disclosure, increases the comprehensiveness and quality of the data obtained by the present methods. Upon lysis and/or digestion of cells and their biomolecules, the embedding particle matrix can facilitate capture of desired biomolecules. For example, in some embodiments polyT oligos attached to the particle backbone may capture released mRNA, or affinity molecules, like aptamers or antibodies, may capture specific epitopes released from cells. The particle properties, such as porosity, may capture molecules larger than a certain size, such as macromolecular DNA that may be sterically trapped within the particle.

In still other embodiments, cell-containing particles can be further processed to label them and their contents. For example, in some embodiments antibodies may bind to specific cells encapsulated in the particles, or fluorescent oligos may hybridize to cellular nucleic acids, such as mRNA, captured in the particles. These labels may facilitate later analysis according to the present disclosure, for example, making specific particles fluorescent for targeted recovery, or providing additional sequences by which to attach barcodes or other useful adaptors for sequencing. Labeled or unlabeled particles may be subjected to further processing, such as activated sorting by FACS or MACS. Alternatively, passive selection may also be performed, for example, by adding to processed particles a chemical that permits specific particles to survive while melting others based on their contents.

As provided herein, fixation and permeabilization based compartmentalization of cells comprises in various embodiments crosslinking fixative, organic solvent, or oxidants. The fixed cells can be permeabilized by treatment with organic solvents, surfactants, or enzymes according to some embodiments of the present disclosure.

To facilitate access to cell based analytes, such as nucleic acids, the fixed and permeabilized cells can be processed. For example, in some embodiments the cells can be processed to reverse transcribed to convert mRNA to cDNA, etc. Optionally, this step can be combined with template switching, ligation, or tagmentation to attach universal adaptors for barcoding and/or sequencing. In some embodiments, genomic DNA can also be tagmented into pieces suitable for sequencing. Tagmentation can be used to fragment and add universal adaptors for barcoding and/or sequencing. Alternatively, in some embodiments cellular analytes can be amplified to facilitate their analysis. F or example, genomic DNA from single cells can be subjected to whole genome amplification to provide multiple copies for later analysis. This could, in some cases, increase the comprehensiveness and quality of the data as provided herein. Biomolecules other than nucleic acids can also be analyzed by staining prior to or after fixation and permeabilization steps in other embodiments. For example, in some embodiments affinity molecules, like aptamers or antibodies, may capture specific epitopes released from cells. These labels may facilitate later analysis, for example, making specific particles fluorescent for targeted recovery, or providing additional sequences by which to attach barcodes or other useful adaptors for sequencing. Labeled or unlabeled particles may be subjected to further processing, such as activated sorting by FACS or MACS.

The processed hydrogels or fixated cells provided herein are, according to some embodiments of the present disclosure, subjected to barcoding to enable scalable single cell sequencing. This can be accomplished with or without microfluidics using a variety of techniques. For example, in some embodiments with microfluidics, single step workflows can be used in which processed particles or cells contain cellular analytes that can be readily barcoded in a single step. For example, processed hydrogels or cells can be introduced into a microfluidic device that randomly pairs them with barcode sequences, such that the barcode sequences are incorporated into the processed analytes, permitting detection by a sequencing instrument. Alternatively, in other embodiments, microwell techniques that function along similar principles can perform this step. This step can also be accomplished using non-microfluidic techniques. For example, in some embodiments processed particles or cells can be subjected to split pool workflows that randomly attach barcodes using a combination of molecular techniques, such as tagmentation, ligation, and polymerase extension. Particle templated emulsification may also be used to randomly pair cell particles with barcodes.

The material resulting from the aforementioned processing and barcoding steps can then be analyzed, using for example sequencing, mass spectrometry, imaging, or other methods known in the art. The barcode information can be used to computationally group together all analytes (e.g., nucleic acids) originating from a single particle, thereby aggregating together information from single cells encapsulated in the particles, and multiple cells such as in paired cell studies.

Thus, as described herein, a method for epigenetic sequencing single cells that use hydrogel-based permeable compartments for partitioning single cells comprises, in various embodiments, one or more of the steps provided below and herein. In the workflow, single cells are individually trapped and lysis within the chemically modified hydrogel microsphere using droplet microfluidics. The hydrogel microspheres are permeable to protein, detergents, and small molecules, but sterically trap genomic DNA. This allows multiple step genome processing required by epigenetic sequencing, while maintaining compartmentalization of each individual genomes. To the genomes trapped in hydrogel microsphere, chemically labelled antibodies or affinity probes targeting specific epigenetic markers are introduced, which specifically bind to the corresponding epigenetic markers and chemically crosslink with the hydrogel network. After genomic DNA fragmentation and washing, only retaining DNA fragments are the ones captured by antibodies or affinity probes. The DNA fragments are then barcoded using microfluidics for sequencing library preparation. This high throughput single cell epigenetic sequencing method can be applied to several epigenetic markers, which include but not limited to, genomic DNA modification (5-methylcytosin, 5-hydroxymethylcytosine, 5-formylcytosine, 5-carboxylcytosine, and 3-Methylcytosine), histone modifications, DNA-transcription factor interaction, DNA accessibility, chromatin conformation, and mRNA-nucleosome interaction.

To facilitate steps involving selective enrichment of epigenetic DNA fragments, the hydrogel can be functionalized. This can be done by chemical modification of the hydrogel precursor or monomer. The capture reagents such as antibody and streptavidin can also be chemically modified to allow crosslinking to the hydrogel network. The functional groups on the hydrogel network and on the capture, reagents need to be compatible for click reaction. To facilitate later steps involving selective enrichment of epigenetic DNA fragments, the epigenetic marker can be modified for capturing. This step can be done by chemical reaction and/or enzymatic reactions. The modifications allow covalent linking of the epigenetic markers to the capture reagents or hydrogel network or non-covalent interaction between the epigenetic marker to the capture reagents. The covalent lines between the epigenetic marker and the capture reagents can be reversable for later PCR amplification and barcoding.

To facilitate later steps involving selective enrichment of epigenetic DNA fragments, the genomic DNA may optionally need to be fragmented. This can be done by using a combination of molecular techniques, such as restriction digestion, fragmentation, and tagmentation. Universal adaptor can be introduced to facilitate barcoding. This can be achieved by adaptor ligation, polymerase extension, nucleotide transfer, or tagmentation. The processed hydrogels can optionally be subjected to barcoding to enable scalable single cell sequencing. This can be accomplished with or without microfluidics using a variety of techniques. For example, with microfluidics, single step workflows can be used in which processed particles contain cellular analytes that can be readily barcoded in a single step. For example processed hydrogels can be introduced into a microfluidic device that randomly pairs them with barcode sequences, such that the barcode sequences are incorporated into the processed analytes, permitting detection by a sequencing instrument. Alternatively, microwell techniques that function along similar principles can perform this step. This step can also be accomplished using non-microfluidic techniques. For example, processed particles can be subjected to split pool workflows that randomly attach barcodes using a combination of molecular techniques, such as tagmentation, ligation, and polymerase extension. Particle-templated emulsification may also be used to randomly pair cell particles with barcodes.

The material resulting from these processing and barcoding steps can then be analyzed, using for example sequencing or other methods. The barcode information can be used to computationally group together all analytes originating from a single particle, thereby aggregating together information from single cells encapsulated in the particles, and multiple cells such as in paired cell studies.

One exemplary workflow is provided as follows:
1. Chemically modification of hydrogel material (agarose as example)
   a. Agarose reacted with reagents to form functionalized agarose (R is functional group with Azide, DBCO, alkyne, tetrazine, TCO, NZ and other groups for click reaction)

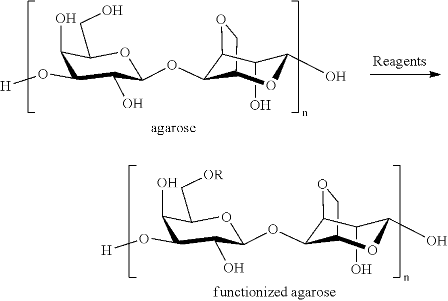

2. Prepare the capture reagents
   a. Antibody, streptavidin, or other capture reagents react with reagents to be modified to introduce functional groups (R is functional group with Azide, DBCO, alkyne, tetrazine, TCO, NZ and other groups that could react with the function group in step 1.a)

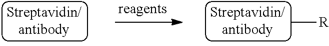

3. Encapsulate single cells in functionalized hydrogel beads.
   a. Controlling concentration
4. Prepare the genomic DNA to allow epigenetic marker capturing or crosslinking to the hydrogel material.
   a. Enzymatic conversion, chemical conversion, or direct antibody capture
   b. Introduce modified capture reagents to crosslink the target analyte with hydrogel beads
5. Prepare the captured DNA to add universal adaptors.
   a. Tagmentation based method
   b. DNase I or other restriction endonuclease, non-specific nuclease based fragmentation followed by ligation
   c. Wash away fragments that are not crosslineds
6. Barcoding processed DNA to enable analysis
   a. Microfluidics
      i. 1 step workflow
         1. 10×, seqwell
      ii. 2 step workflow
         1. MissionBio
   b. Non microfluidics (split pool, Fluent)
      i. Split pool
      ii. Fluent
         7. The resultant material recovered and prepared for analysis
   Sequencing, mass spectrometry, imaging, etc.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a conformation switching probe” includes a plurality of such conformation switching probes and reference to “the microfluidic device” includes reference to one or more microfluidic devices and equivalents thereof known to those skilled in the art, and so forth. It is further noted that the claims may be drafted to exclude any element, e.g., any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. This is intended to provide support for all such combinations.

### Example 1

### Single Cell Epigenetic Sequencing of DNA Comprising a 5-Hydroxymethylcytosine Epigenetic Marker

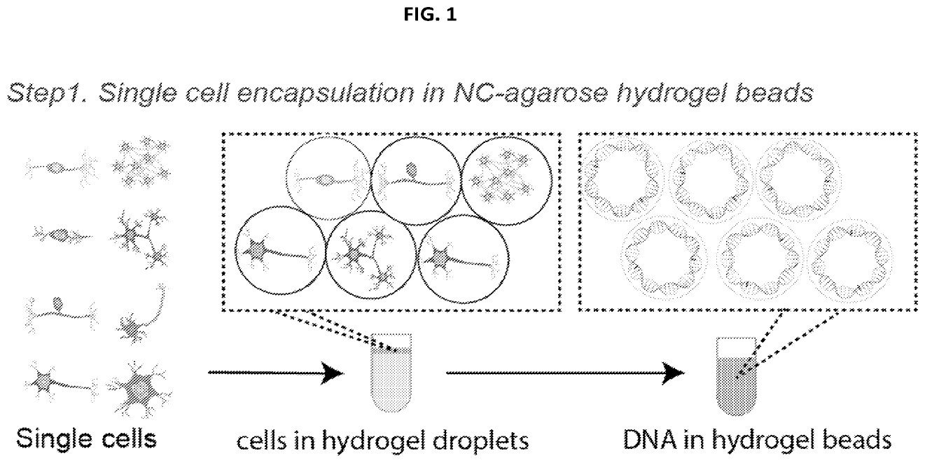

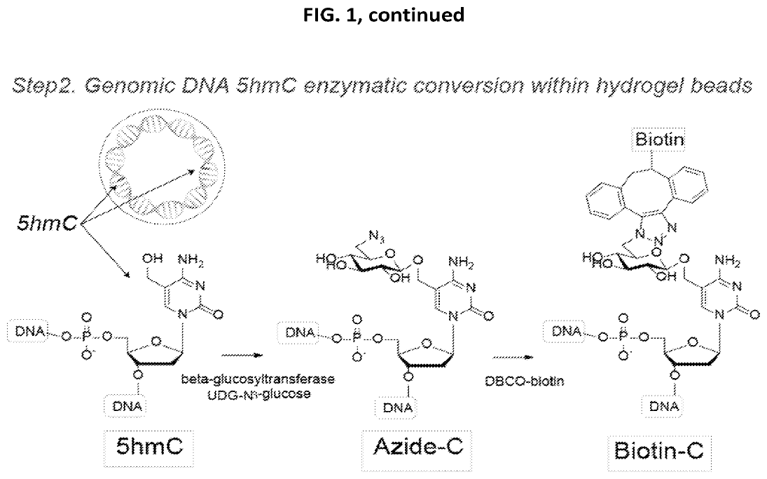

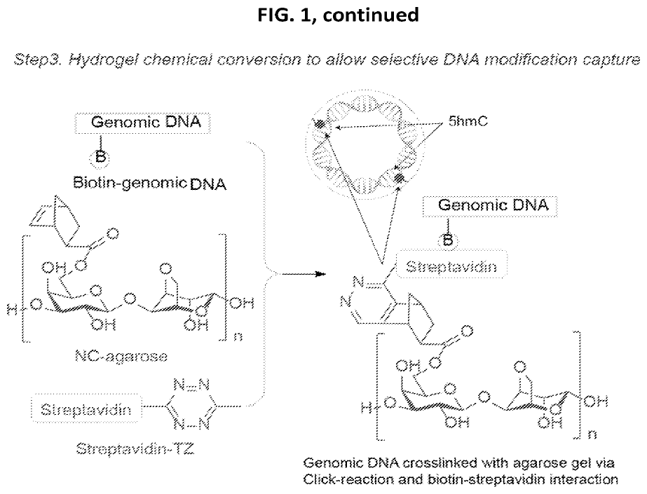

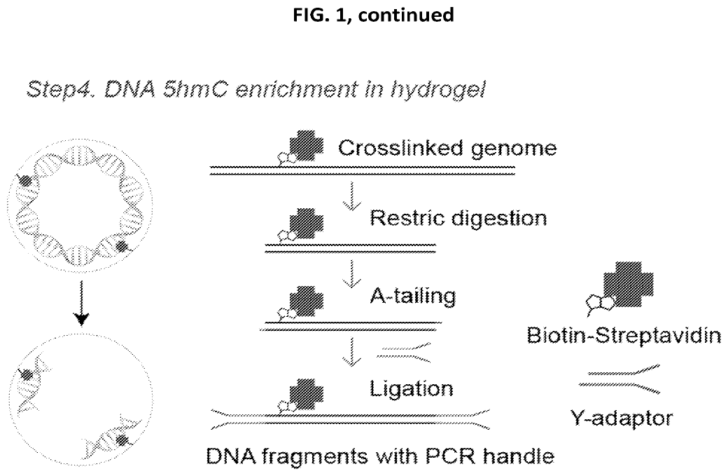

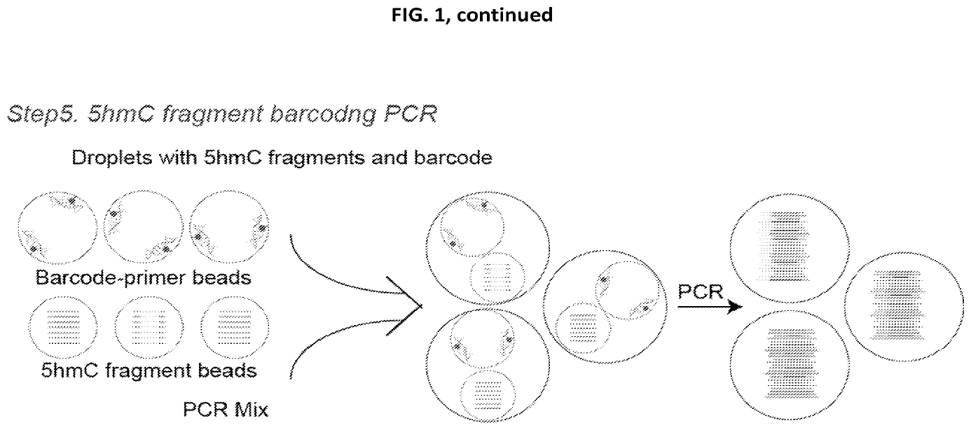

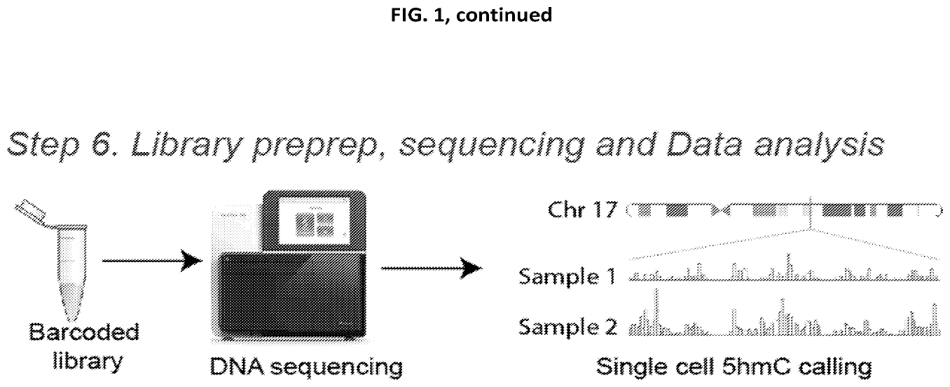

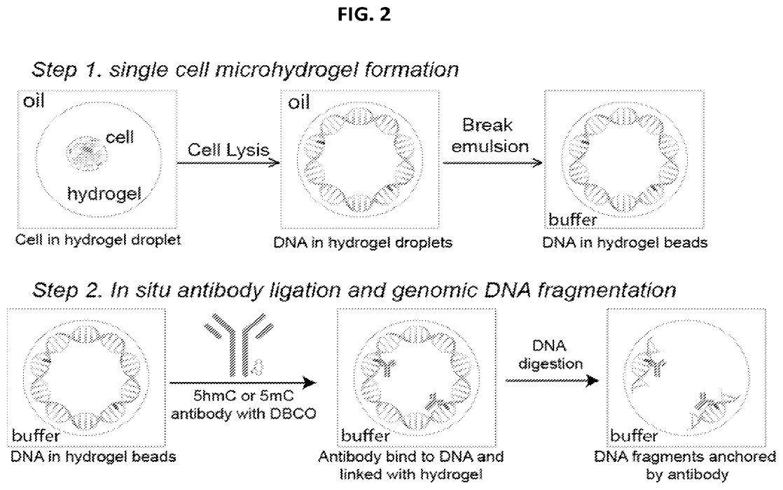

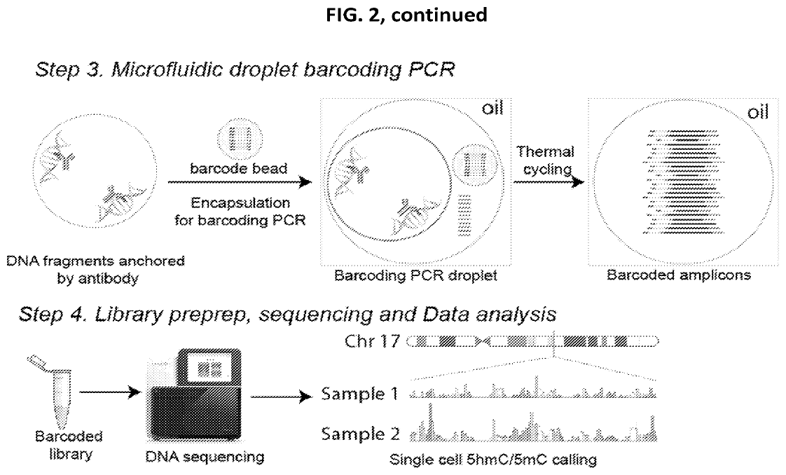

The following Example provides a workflow for sequencing DNA with a 5hmC epigenetic marker. The workflow is also provided in FIG. 1.
1. Synthesize NC agarose.

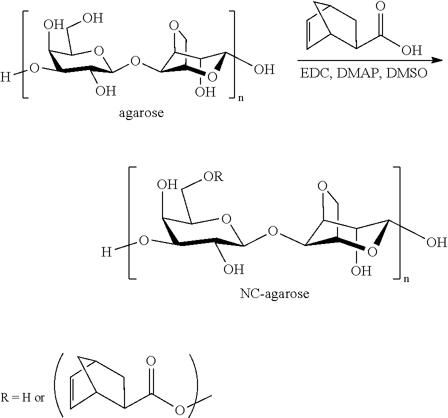

2. Synthesize tetrazine-streptavidin
3. Encapsulate single cells in NC agarose droplets and cell lysis buffer.
4. After cell lysis, heat up the droplet to 90 to denature the DNA and then cool the droplet to allow gelation.
5. Break emulsification with PFO.
6. Wash the beads with PBS-tween buffer
7. Selectively modify 5hmC with biotin
   a. Beta-glucosyltranferase and UDG-N3-glucose to introduce azide-glucosyl
   b. DBCO-biotin to react with azide to introduce biotin
8. Crosslink the biotin-5hmC with hydrogel network using tetrazine-streptavidin
   a. Streptavidin bind with biotin on the DNA
   b. Tetrazine covalently link to NZ-agarose.
9. Prepare the DNA for barcoding.
   a. DNA fragmentation
   b. End repair
   c. Ligation to add universal adaptor
10. Wash the beads to remove non-crosslined DNA fragments
   a. 5hmC containing fragments retained
   b. Fragments without 5hmC are washed away
11. Barcoding PCR.
   a. Microfluidics based (Mission bio, 10×).
   b. Non microfluidcs based (microwell array).
   c. FACS sorting and multiwell plates

### Example 2

### Single Cell Epigenetic Sequencing of DNA Comprising a 5-Methylcytosine Epigenetic Marker

The following Example provides a workflow for sequencing DNA with a 5mC epigenetic marker.
1. Synthesize NC agarose.

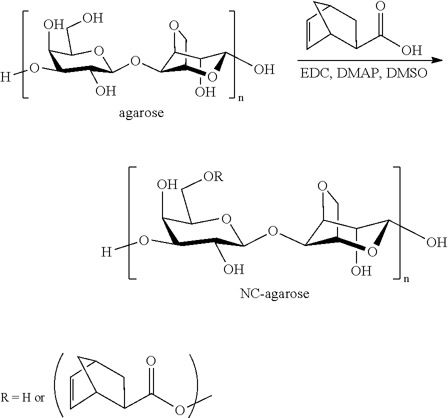

2. Synthesize tetrazine-antibody (Anti-5mC)
3. Encapsulate single cells in NC agarose droplets and cell lysis buffer.
4. After cell lysis, heat up the droplet to 90 to denature the DNA and then cool the droplet to allow gelation.
5. Break emulsification with PFO.
6. Wash the beads with PBS-tween buffer
7. Introduce Antibody to capture the 5mC rich region to hydrogel network
   a. antibody bind to 5mC region
   b. Tetrazine covalently link to NZ-agarose.
8. Prepare the DNA for barcoding.
   a. DNA fragmentation
   b. End repair
   c. Ligation to add universal adaptor
9. Wash the beads to remove non-crosslined DNA fragments
   a. 5hmC containing fragments retained
   b. Fragments without 5hmC are washed away
10. Barcoding PCR.
   a. Microfluidics based (Mission bio, 10×).
   b. Non microfluidcs based (microwell array).
   c. FACS sorting and multiwell plates

The various embodiments described above can be combined to provide further embodiments. All U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

What is claimed is:

1. A method of determining the epigenomic state of a single cell comprising the steps of:
(a) preparing a functionalized hydrogel, wherein said functionalized hydrogel is chemically modified with a functional group capable of binding to a capture reagent;
(b) preparing a functionalized capture reagent, wherein said functionalized capture reagent is chemically modified with a functional group capable of binding to the functional group of the functionalized hydrogel of (a), and wherein said capture reagent is also capable of binding to a nucleic acid;
(c) encapsulating single cells into particles comprising a cell lysis buffer and the functionalized hydrogel of (a) under conditions that allow cell lysis;
(d) preparing a nucleic acid from the encapsulated single cells of (c) under conditions that allow binding of the capture reagent to (i) the nucleic acid, and (ii) the functional group of the functionalized hydrogel, thereby forming a captured nucleic;
(e) preparing the captured nucleic acid of (d) for sequencing, wherein said preparing comprises universal adaptor ligation and barcoding; and
(f) sequencing the captured nucleic acid and thereby determining the epigenomic state of a single cell.

2. The method of claim 1, wherein the hydrogel comprises a polysaccharide selected from the group consisting of agarose, alginate, chitin, chitosan, or hybridize hydrogel material containing a polysaccharide.

3. The method of any one of claims 1-2, wherein the functional group capable of binding to the capture reagent is selected from the group consisting of azide (N₃), dibenzocyclooctyne (DBCO), alkyne, tetrazine (TZ), methyltetrazine, trans-cyclooctene (TCO), cyclooctene, norbornene (NZ), cyclopropene, thiol, bromo, tosylate, maleimide, amine, carboxylic acid and NHS ester.

4. The method of any of the preceding claims, wherein the functional group capable of binding to the functional group of the functionalized hydrogel is selected from the group consisting of azide (N₃), dibenzocyclooctyne (DBCO), alkyne, tetrazine (TZ), methyltetrazine, trans-cyclooctene (TCO), cyclooctene, norbornene (NZ), cyclopropene, thiol, bromo, tosylate, maleimide, amine, carboxylic acid and NHS ester.

5. The method of any of the preceding claims, wherein the capture reagent is selected from the group consisting of an antibody, streptavidin, avidin, and aptamers.

6. The method of any of the preceding claims, wherein the nucleic acid is genomic DNA.

7. The method of claim 6, wherein the capture reagent is capable of binding to an epigenetic marker of the genomic DNA.

8. The method of claim 7, wherein the epigenetic marker is selected from the group consisting of a genomic DNA modification marker, a histone modification marker, a DNA-transcription factor interaction marker, a DNA accessibility marker, a chromatin conformation marker, and a mRNA-nucleosome interaction marker.

9. The method of claim 8, wherein the epigenetic marker is a genomic DNA modification marker selected from the group consisting of 5-methylcytosin, 5-hydroxymethylcytosine, 5-formylcytosine, 5-carboxylcytosine, and 3-methylcytosine.

10. The method of any of the preceding claims, wherein the conditions of (d) that allow binding of the capture reagent to (i) the nucleic acid, and (ii) the functional group of the functionalized hydrogel comprises cross-linking the capture reagent to the hydrogel.
